(19) United States
(12) Patent Application Publication
(10) Pub. No.: US 2009/0326381 A1
(43) Pub. Date: Dec. 31, 2009

(54) SYSTEM AND METHOD TO FACILITATE HEALTH EVALUATION AND MEDICAL DIAGNOSIS OF A HUMAN SUBJECT

(76) Inventors: Yun-e Yuan, Beijing (CN); Xiao-sen Ge, Cedar Knolls, NJ (US); Hong-Juan Li, Beijing (CN)

Correspondence Address: GREENBERG TRAURIG (LV), 3773 HOWARD HUGHES PARKWAY, Suite 400 North, LAS VEGAS, NV, 89169, US

(21) Appl. No.: 12/163,748

(22) Filed: Jun. 27, 2008

(51) Int. Cl. A61B 6/00

(52) U.S. Cl. 600/473

(57) ABSTRACT

A method and system to facilitate health evaluation of a human subject using one or more thermal images of the human subject. The thermal images can be converted into electronic data and subjected to signal processing. Spatial distribution of the data can be calculated and determined to provide energy points that can subsequently be compared to and analyzed with pre-determined meridians and vessels to provide a medical diagnosis of the human subject.

### FIELD OF THE INVENTION

The embodiments of the present invention relate to health and medical systems and methods, more specifically, to a system and method of facilitating health evaluation and providing medical diagnosis of a human subject.

### BACKGROUND

Modern medical imaging technologies including magnetic resonance imaging (MRI), ultrasounds, PET scans and radionuclide imaging have become primary means for diagnosing medical ailments in humans. These imaging technologies are able to provide the organizational structures of the human body but cannot fully reflect or predict functional changes within the human organs. Furthermore, some imaging technologies have proven to be invasive and may cause external and/or internal damage to the patient.

As such, there exists a need for a detection and imaging technology that not only provides the organizational structures of the human body, but one that also allows medical personnel to provide cursory health evaluation and diagnose functional changes in determining an appropriate medical course of action.

### SUMMARY

Accordingly, a first embodiment of the present invention discloses a method comprising: acquiring one or more images of a human subject; defining one or more bone structures of the human subject using the one or more images; identifying one or more parts of the human subject based on the one or more bone structures and the one or more images; and establishing one or more viscera and bowels, meridians and vessels and acupoints of the human subject based on geometric ratios between the bone structures and parts of the human subject. In another embodiment, the method also includes analyzing one or more temperature features from the one or more images for identifying the one or more parts of the human subject. In another embodiment, the method further includes dissecting the one or more images of the human subject into one or more regions to facilitate defining the one or more bone structures of the human subject. The steps as described above can be manually or automatically executed.

A second embodiment of the present invention discloses a computer system comprising: means for defining one or more bone structures of a human subject from one or more images of the human subject; means for identifying one or more parts of the human subject based on the one or more bone structures; and means for establishing one or more viscera and bowels, meridians and vessels and acupoints of the human subject based on geometric ratios between the bone structures and parts of the human subject. Another embodiment of the system also includes means for analyzing one or more temperature features from the one or more images for identifying the one or more parts of the human subject. Another embodiment of the system further includes means for dissecting the one or more images of the human subject into one or more regions to facilitate defining the one or more bone structures of the human subject. In another embodiment, the system also includes means for storing the one or more viscera and bowels, meridians and vessels and acupoints on an electronic medium. The computer system can be automated.

A third embodiment of the present invention discloses a method comprising: acquiring one or more thermal points of a human subject; calculating spatial distribution of the one or more thermal points to provide one or more energy points of the human subject; and comparing the one or more energy points with one or more meridians and vessels of the human subject to provide a health evaluation of the human subject. In another embodiment, the method further includes acquiring one or more thermal images of the human subject using one or more imaging devices, and converting the one or more thermal images into the one or more thermal points of the human subject. In another embodiment, the method also includes performing data processing on the one or more thermal points of the human subject, wherein the data processing includes signal smoothing and improving signal to noise ratio of the one or more thermal points. In another embodiment, the method includes displaying the one or more thermal points as one or more temperature field maps, and analyzing the one or more energy points with the one or more viscera and bowels, meridians and vessels and acupoints to provide a medical diagnosis of the human subject.

The one or more viscera and bowels, meridians and vessels and acupoints can be pre-determined from one or more images of the human subject and can be pre-stored on one or more databases. The one or more energy points include one or more qi (pronounced “chee”) points. The steps as described above can be manually or automatically executed.

A fourth embodiment of the present invention discloses a computer system comprising: means for receiving one or more thermal points of a human subject; means for calculating spatial distribution of the one or more thermal points to provide one or more energy points of the human subject; and means for comparing the one or more energy points with one or more viscera and bowels, meridians and vessels and acupoints of the human subject to provide a health evaluation of the human subject. In another embodiment, the system further includes means for receiving one or more thermal images of the human subject acquired using one or more imaging devices, and means for converting the one or more thermal images of the human subject into the one or more thermal points. In another embodiment, the system also includes means for performing data processing of the one or more thermal points of the human subject, wherein the means for performing data processing includes signal smoothing and improving signal to noise ratio of the one or more thermal points. In another embodiment, the system includes means for displaying the one or more thermal points of the human subject as one or more temperature field maps, and means for analyzing the one or more energy points with the one or more viscera and bowels, meridians and vessels and acupoints of the human subject to provide a medical diagnosis of the human subject. The one or more meridians and vessels can be pre-determined from one or more images of the human subject and pre-stored on one or more databases. The one or more energy points include one or more qi points. In yet another embodiment, the system includes means for storing the one or more meridians and vessels on an electronic medium. The computer systems as described above can be automated.

Other variations, embodiments and features of the present invention will become evident from the following detailed description, drawings and claims.

### BRIEF DESCRIPTION OF THE DRAWINGS

FIG. 1 illustrates a block diagram of a method to facilitate health evaluation and medical diagnosis of a human subject according to a first embodiment of the present invention;

FIG. 2 illustrates an infrared thermal image of the backside of a human subject;

FIGS. 3-4 illustrate three-dimensional spatial distribution thermal maps of the infrared thermal image of FIG. 2;

FIG. 5 illustrates a process flow of a method to facilitate medical diagnosis of a human subject according to another embodiment of the present invention;

FIG. 6 illustrates a process flow of a method to facilitate health evaluation of a human subject according to another embodiment of the present invention;

FIG. 7 illustrates a perspective view of the position fitting step for the health evaluation of the human subject;

FIGS. 8A-8B illustrate perspective frontal and posterior views, respectively, of the spatial distribution thermal flux about a human subject;

FIG. 9 illustrates a perspective view of the various regions of the human subject for purposes of dividing the body into different regions for determining the viscera and bowels, meridians, vessels and acupoints;

FIGS. 10B-12B illustrate perspective views of the process of acquiring the positions or acupoints of the lung Meridian of hand taiyin; and

FIGS. 13B-23B illustrate imaging results of case studies of human subjects utilizing embodiments of the disclosed health evaluation and medical diagnosis system and method.

### DETAILED DESCRIPTION

It will be appreciated by those of ordinary skill in the art that the invention can be embodied in other specific forms without departing from the spirit or essential character thereof. The presently disclosed embodiments are therefore considered in all respects to be illustrative and not restrictive.

Based on temperature distribution of a human body, the embodiments of the present invention allow automatic calculations of inner body core temperatures to be determined using body surface temperatures exclusive of any ambient interference. The results may be displayed on two-dimensional and three-dimensional spatial distribution image maps to facilitate health evaluation and medical assessment of the human subject.

A human body is a source of spontaneous infrared radiation, and can continuously dissipate the infrared radiation to the surrounding environment. Such infrared radiation, energy metabolism, body heat balance and body structure and organization are intrinsically related. The human body produces heat through its metabolic reactions. Heat is a form of energy that can be measured as temperature. The body temperature can be determined by the structure and function of various anatomical parts including blood flow rate, structure of the subcutaneous tissue, activities of the nervous system and so forth.

The human skin is one of the most important places for measuring human heat, wherein the skin blood circulation determines the amount of heat that is provided to the skin. Therefore, any of the factors (physiological, pathological, physical, environmental, emotional, etc.) which influence the blood vessels or skin surface may also affect human body heat and associated infrared radiation. Thus, it is desirous to perform infrared thermal imaging of a human subject under certain conditions and instantaneously capture information about the body and relevant inner organs. Because the temperature at the surface of the skin can be controlled by the physical and emotional environment as well as other non-physiological and pathological factors, infrared thermal imaging and associated analysis of the thermal maps can reflect physiological and pathological changes.

The types of heat capable of being imaged and calculated include surface heat or temperature (shell temperature or skin temperature), superficial heat, deep penetrating heat, internal heat or core temperature, non-planar internal heat source, physiological body structure heat or temperature, heat from blood vessels, organs and cavities, heat from beneath the hair, and other spatial heat or temperature regions. The embodiments of the present invention assume that the human body is an open system capable of naturally maintaining a balanced temperature unless an event has occurred causing an imbalance. Accordingly, traditional Chinese medicine has been incorporated as the homeostatic model of the human body with 3 cavities, 12 meridians and 2 vessels, wherein each meridian is divided into different locations on the body with relations to the various internal organs.

Traditional Chinese medicine focuses not only on discrete components like organic and inorganic substances, proteins, tissues and cells of a human body, it also sees the body as an entity with interconnecting parts that work together to sustain life. Some parts have more energy properties while others have more material properties. Because the interaction between different parts of the body is vital to the individual well-being, it can be sometimes be referred to as the fundamental substances necessary for life having blood, body fluids and “qi” (pronounced ‘chee’). The Chinese character “qi” is the same word for air or gas, and can be interpreted as the “life energy” or “life force” that flows within the human body and the surrounding universe.

There are various types of qi flowing in various directions within a human body. In one example, traditional Chinese medicine believes that “protective qi” provides a human body with protection against evils. The types of evils include environmental factors that can lead to illnesses. In Western context, “protective qi” functions like the immune system, which helps to prevent diseases from occurring or spreading in the human body. One of the advantages of “protective qi” is that traditional Chinese medicine believes it can be monitored by surface temperature. A normal human body should have hot and cold regions evenly distributed and bilaterally symmetrical. When the body surface temperature is too low, it is possible that the person's “protective qi” has weakened and is more susceptible to illnesses including the likes of a cold or flu. Conversely, when the left and right sides of the body are out of balance (e.g., the temperature of left side being higher than the right side or vice versa), it may be indicative of an illness or lack of wellness within the body.

“Protective qi” can also come from the food essence derived by the spleen and stomach. It moves outside the blood vessels and circulates in different areas. Internally, it can be distributed to the diaphragm and scattered about the chest and abdominal cavities. Externally, it moves between the skin and muscles providing the desired protection. “Protective qi” not only guards against illnesses and diseases, it also regulates the sweat glands and pores and provides nourishment for the skin, hair and muscles.

Thermal temperatures can be expressed on the body and cavities including burning heat and five senses of the human head, and can sometimes be used to illustrate thermal temperatures at meridian or channel points based on blood and depth of the heat distribution. The thermal temperatures can also be inferred from oral cavities, skull, limbs, palms, the conception vessel (CV) and governor vessel (GV). Tests have shown that taking Chinese botanical extracts like yinhua forsynthia can further delineate heat distribution, wherein thermal temperature maps can be utilized to illustrate surface body temperature changes on a human body having consumed such extract. In other instances, subjects taking extracts like gypsum, huang lian and the likes have shown a decline in thermal temperatures near the chest area.

In accordance with traditional Chinese medicine principles, meridians are channel points within the human body having characteristics of high electrical potential, low resistance, high sound conduction and high biological conversion of light energy through the migration of radionuclides and associated high temperature circulation with biophysical characteristics. As such, these properties can render infrared thermal imaging with sound, electric, magnetic and nuclear methods advantageous and intuitively objective. In a natural state, a plurality of meridians, vessels and channels with high temperature lines can be distributed throughout the human body. From these meridians, vessels and channels, the dorsal midline governor vessel (GV) has one of the longest lines and one of the highest sensitivity for detection purposes. In one example, AIDS patients may exhibit weaker health condition caused by functional damages to the immune system and the constant state of generating a lower than normal temperature distribution about the dorsal midline governor vessel (GV) and higher than normal temperature distribution about the frontal midline conception vessel (CV). In other instances, higher or lower than normal metabolic heat may be demonstrated in other meridians. Although described as surface or superficial temperatures, many of the meridians, vessels or channels can be several centimeters beneath the surface of the skin. The meridians, vessels and channels may also be described as energy points similar to acupuncture points or acupoints.

The concept behind the essence of traditional Chinese medicine focuses on the universe and energy changes of celestial bodies and its impact on the state of human lives. Earth's climate and the planet's biological rhythm of growth and metabolism are major drivers behind the change. Human physiology and pathology follow basic laws of energy metabolism having different growth cycles and metabolic processes. For example, during summer when the weather is hot, human metabolism and pulses are faster and stronger and fully capable of releasing above-normal amounts of energy. Conversely, during winter when weather is cold, human metabolism and pulses are slower and weaker, and thus, energy and resources need to be absorbed or consumed. Additionally, people that grow up in the mountains exhibit significant differences in physique than those that grow up on the coast because their respective environment exhibits different energy fields.

Research has suggested that studies of desert-like geographical phenomena may lead to drought-like conditions and climate. The cause of this climate formation must have some relation to Earth's rotation and the non-uniform energy distribution of the atmosphere. Thus, to alter a desert climate requires looking beyond mere soil studies and elements missing from the soil, but also environmental management and changes on a global scale must be carried out in order to adequately address the desert climate and associated drought-like condition. The same philosophy can be applied to the theories behind traditional Chinese medicine. When a human tumor is formed and subsequently removed by surgery or other techniques, there is no guarantee that the tumor will not return or that the cancer will not relapse. Only by treating the human body as a whole, including systemic changes in blood, can the cancer or tumor cell be cured. Accordingly, this philosophy forms the basis of the treatment behind traditional Chinese medicine.

As such, the embodiments of the present invention incorporate traditional Chinese medicine standardized assessment capable of being correlated with that of infrared thermal scans to provide health evaluation and medical diagnosis of a human subject. Initially, thermal images of the human subject are acquired to provide surface temperature or superficial heat. Subsequently, the thermal readings can be converted to provide numerical values and subjected to algorithmic calculations to provide inner core temperatures and the corresponding intensity. Accordingly, the derived temperature and intensity readings are compared and analyzed with meridians, channels and vessels of the human subject to provide the health evaluation and medical diagnosis. The meridians, channels and vessels can be established based on geometric ratios of bone structures and portions of the human body, these portions being body parts including arm, leg, head, torso, etc. as well as visceral organs including lung, heart, organs of the digestive system, etc. The meridians, channels and vessels of the human body can also be determined based on ratios of the bone structures to other parts of the body. Although infrared thermal scans are disclosed, it will be understood by one skilled in the art that other imaging techniques including x-rays, MRIs and CT scans may be incorporated for the health evaluation and medical diagnosis.

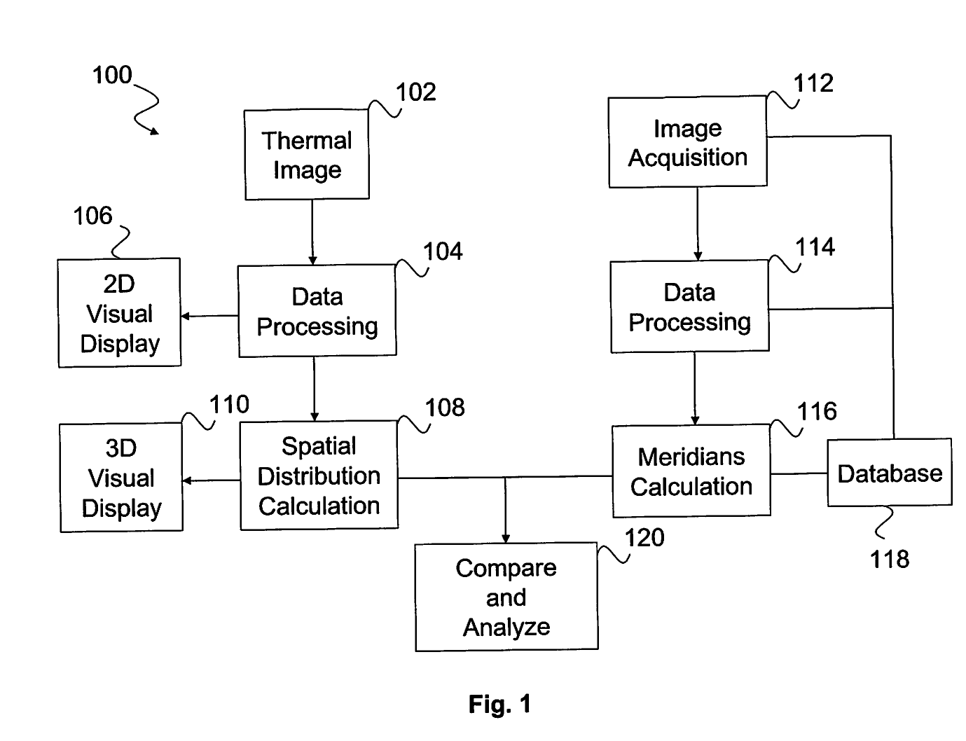

Reference is now made to FIG. 1 illustrating a block diagram of a method of facilitating health evaluation and medical diagnosis of a human subject 100 according to a first embodiment of the present invention. One or more thermal images 102 of the human subject are acquired using any suitable infrared imaging system or method including electronic optical imaging devices. The infrared images 102 include thermal, infrared or thermographic images. The body of the human subject can be positioned (e.g., standing up, sitting down, prone or supine) as necessary for scanning and acquisition purposes. In one example, the infrared hardware includes an un-cooled focal-plane array detector with 320×240 pixel image resolution having electronic focusing capability, acquisition frame rate of 30 frames per second, temperature resolution of 0.08° C., temperature measurement accuracy of ±0.5° C., and spectral responses of 8-14 microns. The thermal images 102 can also be collected using other suitable hardware and software including infrared cameras, imaging sensors and detectors or other imaging devices as can be appreciated by one skilled in the art.

Once acquired, the thermal image 102 can be stored in electronic format on one or more databases (not shown). Instead of acquiring the thermal image 102 in real time, it may also be provided from an external source. Whether scanned, uploaded or manually acquired, the thermal image 102 can be processed using a computer system 104 with known hardware and software components to deliver pixel information in a two-dimensional temperature field data and subsequently provided on a two-dimensional visual display 106. In other words, the thermal image 102 may be converted into one or more thermal points capable of being displayed on a two-dimensional thermal map 106. In one instance, the two-dimensional thermal display 106 provides the surface temperature of the human subject as acquired from thermal imaging 102. A two-dimensional thermal map 106 allows a user to pinpoint with high accuracy an exact surface temperature of the human subject on an X-Y plot 106 using a computer keyboard and mouse as can be appreciated by one skilled in the art. It will also be appreciated that the computer system capable of processing 104 the thermal image 102 can include microprocessor, monitor, hard drive, operating system and other hardware and software as understood by one skilled in the art.

Additionally, the data processing 104 can also involve converting the thermal data 102 into suitable electrical signals and/or other electronically readable format. Additional filtering and/or modulation may be incorporated as necessary. The electronic signals can also be quantified and converted to digital signals with multimedia image processing techniques 104. Although the data processing step 104 as described involves manipulating thermal images 102 acquired from thermal scanning techniques, it will be understood by one skilled in the art that the thermal images 102 need not be acquired but may be provided in electronic format. In other words, the data processing 104 can take place on thermal images 102 that are supplied in other electronically executable format for manipulating and processing purposes 104. Furthermore, the thermal image 102 may be subjected to signal manipulation to smooth the thermal data 102. The data processing 104 can also be performed on the visual display 106 to provide a smoothed two-dimensional temperature display 106. In the alternative, data processing techniques 104 may also include methods of improving the signal to noise ratio of the acquired thermal information 102. Other signal processing techniques as can be appreciated by one skilled in the art may also be utilized.

The processed data 104 are subsequently subjected to mathematical algorithms to provide spatial distribution values 108. In this instance, the heat flux and temperatures beneath the skin of the human body are derived and extracted using the measured thermal data 102 (e.g., surface of the skin). Inverse heat conduction modeling allows for the generation of temperature data points beneath the surface of the skin based on surface thermal imaging information. By knowing the temperature at the surface of a human body, the temperature and intensity at various depths of the body can be calculated using heat equations and heat conduction models, which allow the temperature and intensity beneath the surface of the skin to be derived. The inverse processing can utilize mathematical equations, functions and relationships to provide the spatial distribution 108 as necessary.

In one example, the mathematical functions and algorithms for deriving the core or inner body temperature at a certain depth and intensity are set forth below. Based on the theoretical analysis of heat conduction equation and the thermal conditions of the physical model, the heat conduction equation being a second-order scalar linear ordinary differential equation and can be given by Equation (1).

∇·k_{eff}∇T(x, y, z)+wρc[T₀(x, y, z)−T(x, y, z)]+Qₘ(x, y, z)=0 (1)

Wherein (x, y, z) space is the calculated Cartesian coordinate system, ∇ is the Hamiltonian operator, k_{eff} is the effective thermal conductivity, T(x, y, z) is the three-dimensional temperature field function, w is the volumetric blood flow rate, ρ is the blood density, c is the specific heat capacity, T₀(x, y, z) is the initial temperature of the blood flow, and Qₘ(x, y, z) is the metabolic heat function of the region of interest.

Using the surface temperature from the thermal measurement, we can calculate a defined temperature and heat distribution using Equation (1) from above. In this instance, the surface temperature as provided by the infrared imaging can be set as the boundary condition in order to solve by direct numerical simulation for the internal body temperature and heat distribution therein. The numerical solution involves applying a Fourier Transform for spectrum space in solving differential equations, the solution for the spectral space being given by Equation (2).

$$T ( p , q , r ) = \frac{w ρ {cT}_{0} ( p , q , r ) + {Q}_{m} ( p , q , r )}{w ρ c + {k}_{eff} {ω}^{2}} \qquad ( 2 )$$

Wherein ω=\sqrt{p²+q²+r²} and (p, q, r) are the wave number and space corresponding to the Fourier coordinate system (x, y, z), respectively, while T(p, q, r), T₀(p, q, r) and Qₘ(p, q, r) are Fourier transformed spectra.

This approach avoids the difficulties associated with difference and finite-volume format derivations. In order to address the weakness of cyclical conditions, the calculations at the boundaries must be treated accordingly. Combining Equations (1) and (2), the final solution for the actual physical model is given by Equation (3).

$$T ( x , y , z ) = \frac{{l}_{x} {l}_{y} {l}_{z}}{8 {π}^{3}} \int \int \int \frac{w ρ {cT}_{0} + {Q}_{m}}{w ρ c + {k}_{eff} {ω}^{2}} {e}^{- i px} {e}^{- i qy} {e}^{- i rz} d Ω + C {\sum }_{i = 1}^{3} {A}_{i} {e}^{\sqrt{\frac{w ρ c}{{k}_{eff}}} {x}_{i + {ϕ}_{i}}} \qquad ( 3 )$$

Wherein lₓ, l_{y}, l_{z} are the lengths of the computational domains x, y, z, respectively, Ω being the size of the computational domain, and Aᵢ, φᵢ, C being parameters to be determined based on boundary conditions and other given or set conditions.

The thermal conductivity of the regional space and surface at the border can be satisfied by Equation (4).

$${k}_{eff} \frac{\partial {T}_{s} ( x , y , z )}{\partial n} = {h}_{eff} ( x , y , z ) \cdot [ {T}_{s} ( x , y , z ) - {T}_{a} ( x , y , z ) ] + {Q}_{eff} ( x , y , z ) \qquad ( 4 )$$

Wherein Tₛ(x, y, z) is the medial edge interface for calculating the temperature distribution domain, n is the perpendicular vector to the interface of various border points, h_{eff}(x, y, z) is the convection heat transfer coefficient, Tₐ(x, y, z) is the lateral boundary surface temperature of the environment, and Q_{eff}(x, y, z) is the evaporation of the thermal dissipation.

Adopting the boundary temperatures results in Equation (5).

$${T}_{s} ( x , y , {z}_{0} ) = {C}_{1} {e}^{\frac{{h}_{eff} ( x , y , {z}_{0} )}{{k}_{eff}} z} + {T}_{a} ( x , y , {z}_{0} ) - \frac{{Q}_{eff} ( x , y , {z}_{0} )}{{h}_{eff} ( x , y , {z}_{0} )} \qquad ( 5 )$$

Wherein z₀ is the boundary condition in the z direction and Tₛ(x, y, z₀) can be utilized for solving the coordinates in the different directions.

Returning now to the calculations for the internal domains and regions using the equations above, after having determined the human body's internal temperature and heat distribution, the information can subsequently be displayed on a three-dimensional thermal map 110. In one instance, the three-dimensional thermal map 110 includes the location and depth of the heat distribution and any corresponding hot spots. The depth, intensity and location of the heat 108 can be compared and analyzed with corresponding anatomical locations and structures in accordance with traditional Chinese medicine principles, which will become more apparent in subsequent figures and discussion.

Additionally, the spatial calculations 108 can also be displayed as a three-dimensional spatial distribution map 110 similar to that of the two-dimensional thermal map 106, with the ability to focus on regional temperatures and determine rates of metabolic changes within the human body. The three-dimensional spatial distribution map 110 may also be enlarged and rotated to provide pinpoint accuracy of each temperature value on the human subject including the depth, intensity and location of the thermal values in accordance with the spatial distribution calculations 108. The colored spatial distribution thermal maps 110 can also be three-dimensional with improved graphics capability and high accuracy. In other instances, the spatial distribution 108 can also be provided as digital signals in picture and/or image formats for analysis and assessment purposes. By examining the spatial distribution map 110 using standardized rules and assessment techniques, any internal body dysfunctions arising from relative changes in thermal radiation values can be quickly determined with high sensitivity. Other anomalies can also be detected by making side-by-side comparisons of normal versus abnormal spatial distribution maps 110 or thermal maps 110 taken over a period of time. The visual display feature 110 can be especially beneficial when there are clear pathological changes, small human body cells and associated cell metabolism.

In other instances, the spatial distribution 108 can be calculated using diffusion, regression or other numerical modeling algorithms. The spatial distribution 108 can also be derived using other mathematical algorithms. The result of the spatial distribution 108 provides one or more energy points based on its spatial location (e.g., depth and intensity). In other words, the temperature at the surface of the human body as provided from the thermal scans 102 may be utilized to project temperatures beneath the skin (e.g., temperatures at 1.5 to 2.0 cm beneath the surface of the skin). These in-depth points may be correlated with meridians and channels 116 and defined as one or more energy points or acupoints about the human body. To produce the one or more energy points, the spatial distribution 108 can include analysis with mathematical expressions including the heat equation and heat transport equation. The depth and intensity of the energy points can also be calculated using the equations described above in combination with other energy density and linear superposition principles. The one or more energy points may also be derived using other suitable spatial distribution functions and algorithms as can be appreciated by one skilled in the art. It will be appreciated by one skilled in the art that the previously described systems and methods can be manually or automatically executed.

In addition to making the spatial distribution calculations 108 and determining the corresponding energy points (e.g., heat thermal points at different depths and intensities), one or more meridians, channels and vessels of the human body 116 must also be identified and defined in order to provide the health evaluation and medical diagnosis 120 in accordance with traditional Chinese medicine theories. The ratio of bone structures to various parts of the human body can be utilized for deriving the meridians, channels and vessels 116. In other words, the process 114 is adaptable for people of all shapes and sizes whether they be fat, skinny, tall or short. Further, the meridians and channels derivation processes 114, 116 are adaptable for both men and women.

Still referring to FIG. 1, a thermal image 112 is acquired to determine the meridians, channels and vessels (e.g. acupoints or acupuncture points) of a human body using similar means as those described above. Besides thermal images 112, x-rays, CT scans, MRIs and other imaging techniques may be incorporated and the corresponding images 112 provided. Once an image has been acquired or provided to the system, the image 112 can be processed 114 using location algorithms and multiple sub-level algorithms to provide the desired meridians, channels and vessels 116. First, a two-dimensional profile of the human subject can be obtained from the image 112. The profile can be the front, back or side views of the human subject. The image 112 can also include that of the human subject in various poses and positions. Once obtained, characteristic points of the human body can be identified whereby the body can be further dissected 114 into multiple parts with the main portions being the chest and torso area. The characteristic point can include an arm, leg, head, torso or any other part of the human body.

Next, the bone structure of the human body can be determined 114 based on the length and location of each bone in relation to other parts of the body. Geometric ratio of the bone structure can be correlated to parts of the body like the clavicle or pubis. In other instances, special points 114 can be determined by judging the thermal temperature features like the armpit because of its relatively high temperature compared to other regions of the body. Other special points including the navel and pubic area may also be utilized. In accordance with traditional Chinese medicine principles, the meridians, channels and vessels of the human body can be determined based on geometric ratios among the bone structures, special points, other thermal features and various parts of the human body. It will be appreciated by one skilled in the art that the method and associated computer system as described above can be manually or automatically executed.

In one example, the “san jiao” is a traditional Chinese medical concept that has been translated as “triple heater,” “triple warmer (three warmers)” or “triple burner (triple metaboliser).” The san jiao meridian is located from the fingernail of the ring finger, up the outside center of the hand and arm, behind the top of the shoulder above the shoulder blade, up the neck to behind the ear, up and around the base of the ear to the top and front, then on the outer tip of the eye brow. The san jiao also occupies the trunk of the body and can be considered in three separate compartments: the upper compartment is from the chest to the diaphragm, which is associated with respiration; the middle compartment is from the diaphragm to the umbilicus, which is associated with digestion; while the lower compartment is from the umbilicus to the top of the pubic bone, which is associated with excretion.

In this instance, to determine the san jiao meridian 116, a thermal image 112, as provided in electronic format, is first analyzed. The thermal profile 112 can be received by a computer system with associated processor, hardware and software as can be appreciated by one skilled in the art. Once executed, one or more bone structures of the human subject can be defined from the thermal image 112 by dissecting the human body into discrete regions for analysis and processing purposes. In this example, the bone structure of the ringer finger, arm, shoulder, head as well as the trunk and torso of the body can be measured and determined 114 using software algorithms and other mathematical functions. Special features including the clavicle and navel areas can be defined along with other thermal features including the armpit and pubic area. Using the thermal data and traditional Chinese medicine principles and theoretical models of the san jiao meridian 116, it is possible to subsequently correlate and calculate the acupoints representative of the san jiao meridian 116 that are specific to the particular human subject. The geometric correlation can also incorporate other parts of the human body including visceral organs, lungs, heart, organs of the digestive system, etc. Once the san jiao meridians, channels and vessels 116 have been determined, they may be condensed into one or more energy points or positions for analytical and diagnostic purposes. The energy points can accordingly be provided for comparison and analysis purposes with the spatial distribution result 108 described earlier.

Additionally, the meridian points can be generated, compiled and stored on a database system 118. Furthermore, other meridians, channels and vessels 116 in accordance with traditional Chinese medicine theory may also be stored on the database 118. In other embodiments, the meridians, channels and vessels 116 can be patient specific or derived from a collection of patients to generate a standardized or proximate model of meridian points or channels 116 based on weight, height, age and sex of the human subject. Like the spatial distribution thermal points 108, the calculated meridians and corresponding energy points 116 may also be provided in three-dimensional visual displays (not shown) and super-imposed with three-dimensional color thermal maps 110 for comparison and analysis purposes.

The database 118 for storing the meridian calculations 116 may also include information about the thermal images 112 as well as information about the doctor and patient. The database 118 may also include patient history charts including test results, list of symptoms and possible diagnosis. The types of image information 112 that can be stored on the database 118 may include image identification, patient identification, patient scan identification, date the thermal images were acquired and parts of the body corresponding to the thermal images. The doctor information may include the doctor's identification number, name, sex, age, clinic location, phone number as well as other relevant information. The patient information may include the patient's identification number, name, sex, age, occupation, address, phone number, attending physician's identification and number, disease information, progress and prognosis. Additional information including the description and history of the disease may also be stored on the database 118.

Although not shown, it will be understood by one skilled in the art that the spatial distribution energy points 108 and corresponding thermal images 102 and data processing 104 may also be stored on the same database or other databases, and can be accessed by any suitable computer system with associated hardware, software and processor. The data as stored on the database 118 can also be accessed via the Internet or other suitable electronic network system as can be appreciated by one skilled in the art. Also, although the thermal images 102, 112 as described above can be automatically provided by known scanning techniques, the thermal temperature data may conceivably be manually inputted from an external source. In other words, images 102, 112 may be acquired and uploaded to the system for analytical and diagnostic purposes.

Once the spatial distributions 108 and the meridians, channels and vessels 116 have been determined and provided, the two sets of information can be compared and analyzed 120 to provide health evaluation and medical diagnosis of the human subject. Initially, the health evaluation 120 involves a preliminary examination of the human subject to determine if his or her body is in balance. Based on traditional Chinese medicine theory, a human body is in balance if the right-hand and left-hand sides are substantially symmetrical in terms of heat distribution and that there are no hot spots. The health evaluation 120 can also be carried out using three-dimensional spatial distribution thermal maps 110 for a cursory examination. In this instance, the spatial distribution energy points 108 can be superimposed with those of the meridians 116 to determine if there is a major imbalance between the left-hand and right-hand sides of the body. The analysis 120 can be further carried out to provide a medical diagnosis, which uses traditional Chinese medicine principles, namely the meridians, channels and vessels theory to determine the flow of “qi” based on the spatial distributions energy points 108.

To provide a medical diagnosis 120, the heat information from the spatial distribution 108 can be combined with the acupoint information from the meridians 116. The spatial distribution 108 is likely to provide thermal flux at certain depth and intensity as derived from the surface temperature. In most instances, the meridians and channels 116 are typically about 1.5 to 2.0 cm beneath the surface of the skin. The analytical process allows a user to correlate the spatial distribution energy points 108 to the acupoints of the meridians 116 to facilitate a medical diagnosis of the human subject. In a first instance when channels 116 are healthy, there should be strong qi or energy flow and there should be no “overflow” or blockage of the channels 116. As a result, the channel structures 116 are not “visible” because there are no hot spots. The spatial distribution energy points 108 are not separate and distinct and there is continuous, harmonious temperature flow or pattern with no risk of the channels 116 “filling up.” When channels 116 are not healthy, there is weak qi or energy flow resulting in poor flow and possible blockage. As a result, there is no place for the built up energy or qi to flow other than through the blood vessels and other parts of the skin or body. Because the heat cannot be dissipated or readily spread out, it then travels to the surface of the body by thermal conduction. Thus, an unhealthy individual usually has a higher body temperature than a healthy individual and corresponding core heat at various spatial distributions energy points 108. The trapped heat flux may also affect other organs of the body causing additional ailments or illnesses.

Also, with an unhealthy individual where there is blockage, traditional Chinese medicine dictates that meridians and channels 116 have a fixed direction of flow. For instance, there are meridians and channels 116 that flow from the chest through the acupoints and out to the thumbs. Others channels 116 can flow from the leg or tip of the leg or the fingers toward the chest. In other words, the various qi or energy flows surrounding a human being are directional. If there is blockage of the channels 116, there is an increase of energy building up in the direction being blocked. For example, if there is blockage of energy flow from the heart to the fingers where the fingers are blocked or show signs of blockage, energy build up occurs near the heart such that a medical assessment 120 of the ailment can be provided near the fingers. In other instances, blockages may result in overflow whereby thermal flux may increase above normal or average. Basic laws of thermodynamic dictate that the heat flux flows from high to low. Surface temperature is usually lower than the core temperature of the body. Ambient temperature is typically at about 23° C. while that of the internal human body is about 40-41° C. If there are body organs with abnormal capacity, the human body cannot dissipate the build up of heat and it passes the heat onto the surface as can be understood by general concepts of thermodynamics.

Based on the thermal distribution about a human body, automatic calculations and analyses can be carried out to determine the body's core temperatures and associated spatial distribution by using the surface temperature of the body and excluding any ambient interference. The derived three-dimensional images of metabolic or spatial distribution heat map may be used to assess the health condition of the individual as well as provide medical diagnosis for such individual. In other embodiments, the derived spatial distribution thermal map may be superimposed over the predetermined meridians and channels to provide the health or medical assessment.

Infrared imaging systems having optical electronics are able to gather infrared radiation from human subjects through filtering, modulating and converting photoelectric signals into electronic or digital signals. With multimedia image processing technology, the temperatures of the human body can be further provided in 2D or 3D colored thermal maps. At the same time, dedicated analytical software application for analyzing the temperatures can display the analyzed temperatures in the form of electronic images. By comparing the normal and abnormal states of the spatial distribution thermal maps using specific laws and measurement methods corresponding to reconstructing parts of the body by the intensity of the cell metabolism, thermal radiation values may be measured whereby internal dysfunctions with 0.05% relative changes in heat radiation can be measured in a matter of minutes, and whereby high sensitivity of the system may lead to early detection of small pathological tissue changes in greater than 80% of the patients.

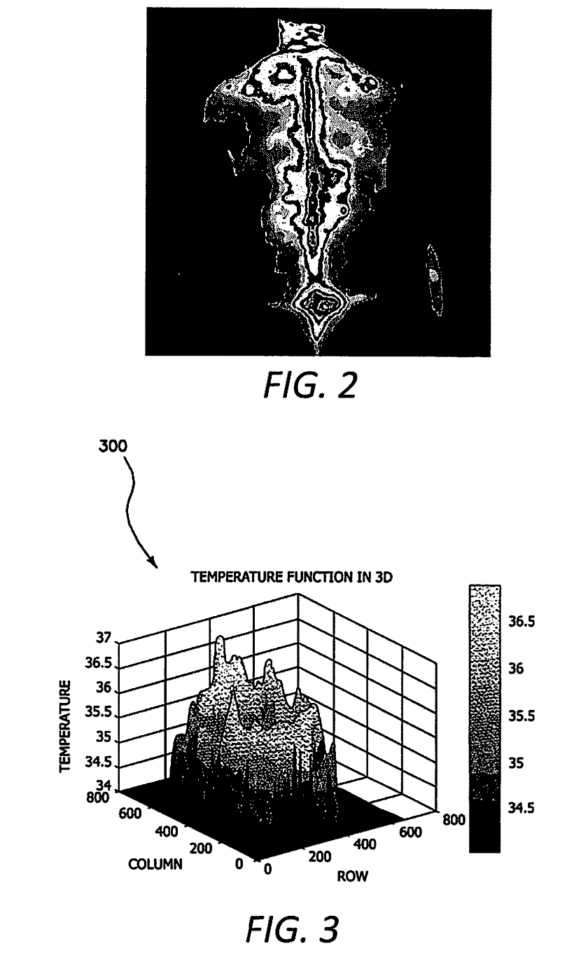

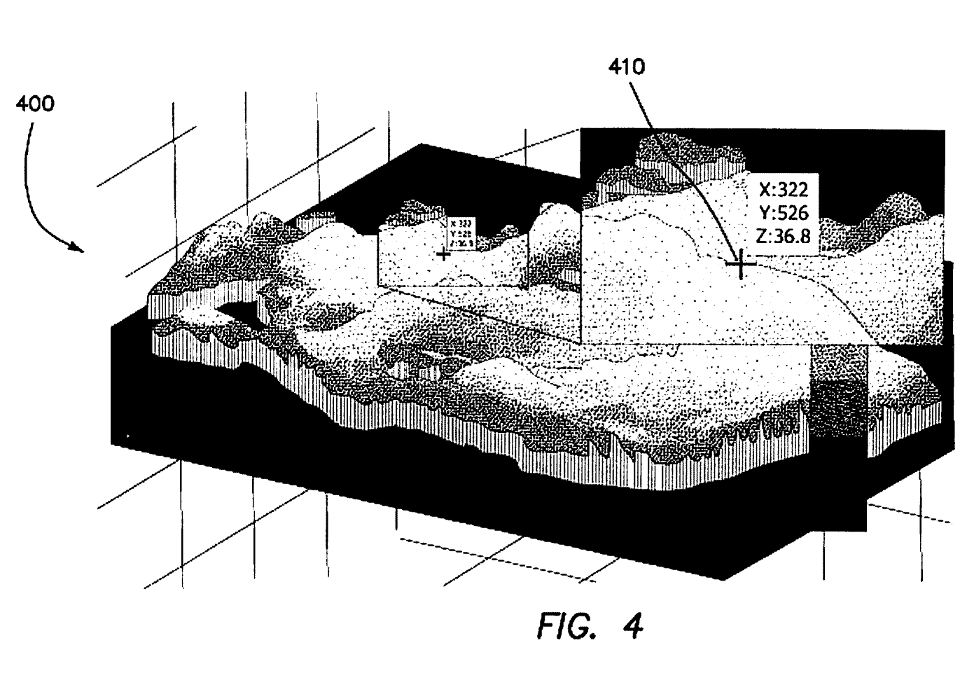

Reference is now made to FIG. 2 illustrating an infrared thermal image 200 as acquired using the electronic and optical devices described above. The thermal image 200 is that of the backside of a human subject. FIG. 3 illustrates a perspective view of a three-dimensional spatial distribution thermal map 220 after the temperature data 200 have been processed and subjected to the spatial distribution calculations discussed above. In other instances, a two-dimensional infrared temperature map in plane coordinates (not shown) may likewise be converted into a three-dimensional thermal map 300 with the ability to focus on and display regional temperatures and rates of change. FIG. 4 illustrates yet another perspective view of a three-dimensional spatial distribution heat flux map 400 whereby an inset window 410 illustrates the ability to provide a close-up of the image 400 with pinpoint accuracy. In this instance, the temperature at the point as identified by the mouse crosshair has a temperature of 36.8° C.

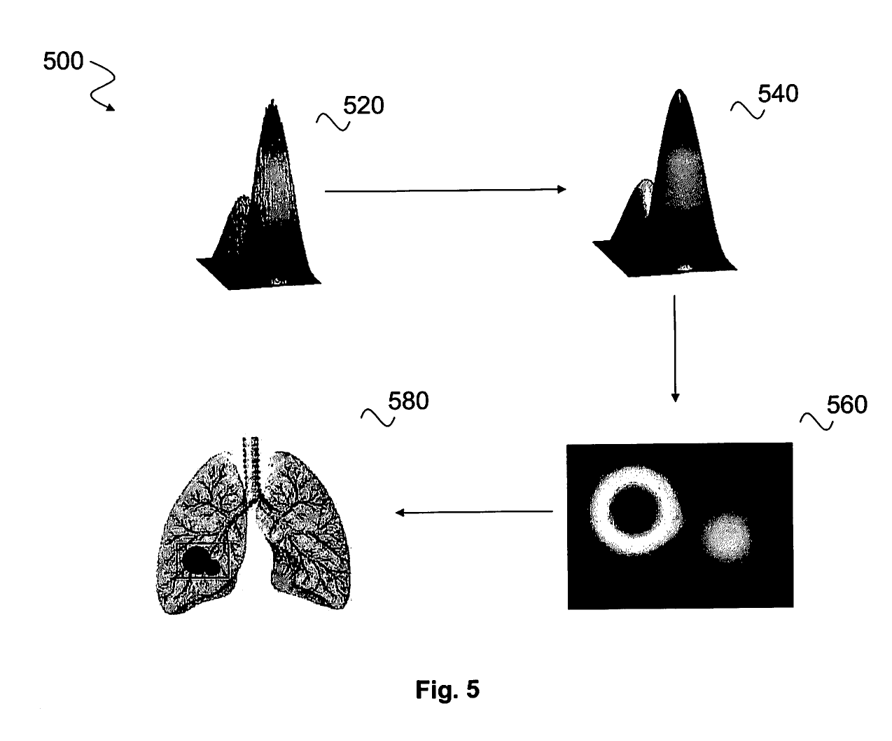

Reference is now made to FIG. 5 illustrating a process flow of a method to facilitate medical diagnosis of a human subject 500 according to another embodiment of the present invention. In this embodiment, an initial data acquisition step 520 allows an infrared image of the human subject to be acquired and provided in a three-dimensional visual display 520. The acquisition of the image and the method of display can be carried out in similar fashion to those described above. After the data has been acquired, a signal smoothing step 540 may be carried out on the acquired data 520 for smoothing or quality handling of the information. Like above, other techniques of smoothing the signal 520 may be carried out. The processed thermal data 540 can subsequently be combined with the physical model of the human subject during a fitting and comparison step 560. The physical model of the human subject includes identification of bone structures and internal organs as well as meridians, channels and vessels that may be determined based on traditional Chinese medicine theory. Once the fittings and comparisons 560 have been made, the information may be superimposed over the particular portion of the human body during an identification and localization step 580. Additional analysis and medical diagnosis of the possible ailment or disease can also take place in conjunction with traditional Chinese medicine principles.

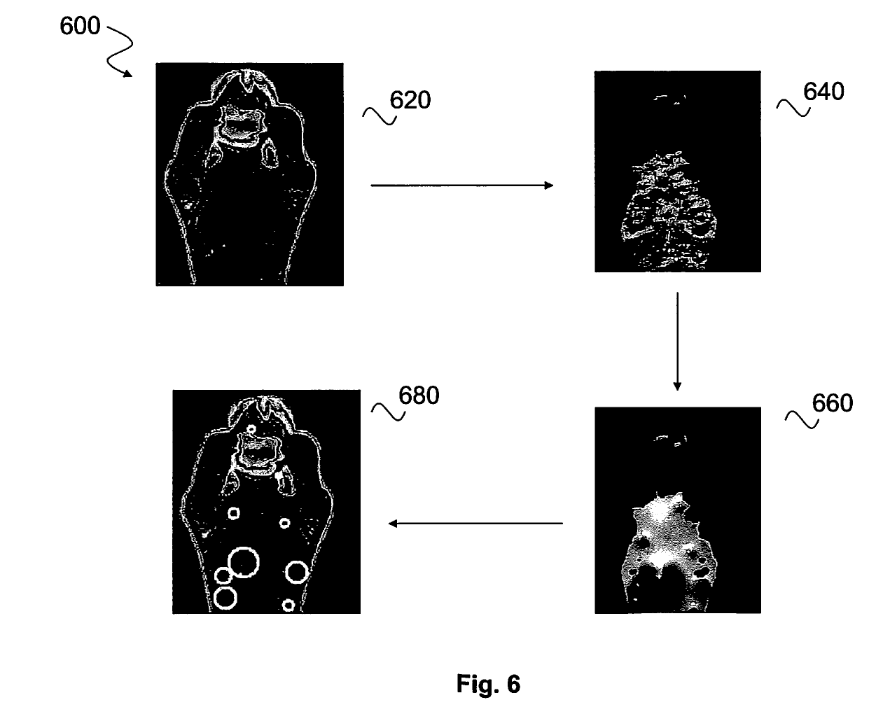

Reference is now made to FIG. 6 illustrating a process flow of a method to facilitate health evaluation of a human subject 600 according to another embodiment of the present invention. In this embodiment, an initial data acquisition step 620 allows an infrared image of the human subject to be acquired and provided in a two-dimensional visual display 620. The acquisition of the image and the method of display can be carried out in similar fashion to those described before. After the data has been acquired, a spatial distribution calculation step 640 can be carried out on the acquired data 620 to determine the depth and intensity of the thermal radiation. The spatial distribution calculation 640 takes into account the infrared temperature acquired from the surface of the human body and accounting for such surface temperature for any ambient interference. The thermal surface temperatures can then be subjected to mathematical algorithms and functions like the heat transport equation and heat conduction equation to provide temperature readings at different depths and intensities.

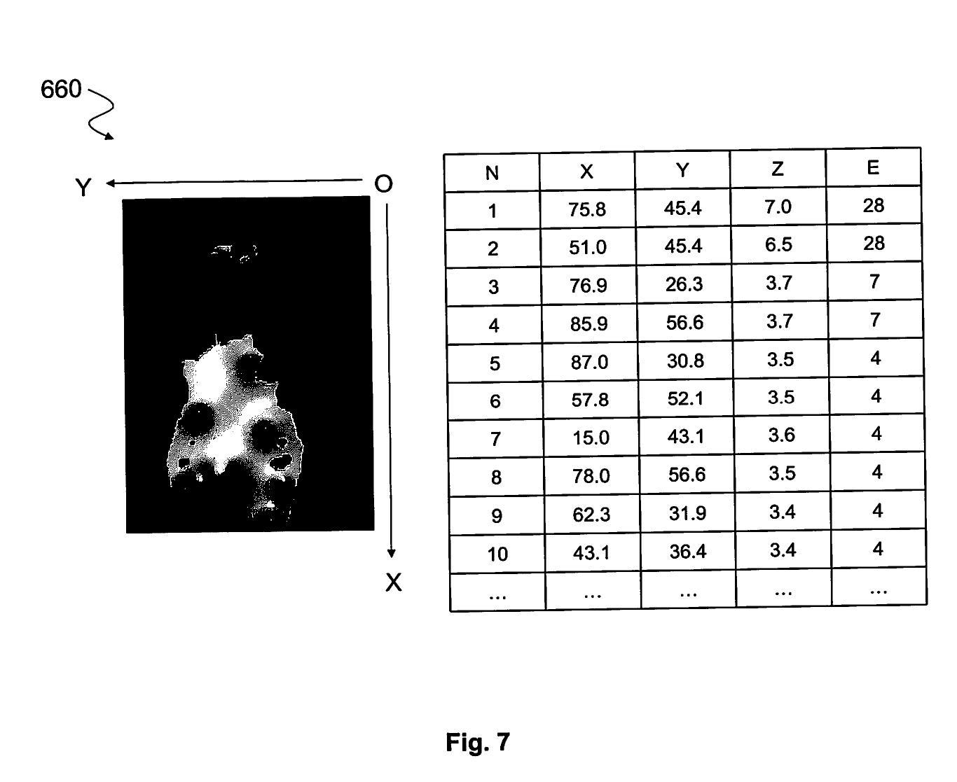

Once the spatial distributions heat flux 640 have been determined, they can be combined with the physical model of the human body during a position fitting step 660. The physical model of the human subject includes identification of bone structures and internal organs as well as meridians, channels and vessels that may be determined based on traditional Chinese medicine theory. The position fitting step 660 can be best shown in FIG. 7, where the temperature readings at different depth (Z) and intensities (E) can be identified and correlated to specific locations (X, Y) with respect to an origin. Returning now to FIG. 6, once the temperature readings have been identified and fitted to portions of the human subject, any hot spots may be highlighted during a define and identify step 680. This information may be super-positioned over the particular portion of the human body as identified during the position fitting step 660. As the define and identify step 680 illustrates, in looking at the left and right sides of the human subject, any imbalance can be quickly determined to provide a cursory health evaluation. Additional in-depth analysis and medical diagnosis of the possible ailment can be further carried out as necessary.

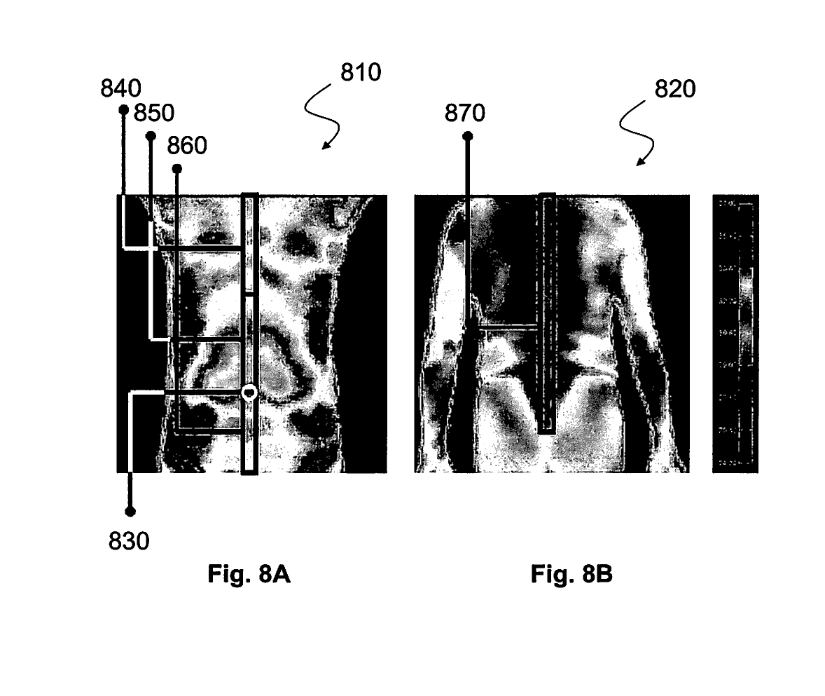

Reference is now made to FIGS. 8A-8B illustrating perspective frontal 810 and posterior 820 views of the spatial distribution thermal flux about a human subject. As shown in the figures, the frontal thermal image 810 is able to provide an upper conception vessel 840 with an average temperature of 33.05° C., a middle conception vessel 850 with an average temperature of 30.15° C. and a lower conception vessel 860 with an average temperature of 32.80° C. The umbilical temperature 830 in this instance is one of the higher values at an average of 35.10° C. Additionally, the du meridian 870 as viewed from the posterior 820 can provide an average temperature of 34.15° C. These figures include the benefits of superimposing or super-positioning the derived spatial distribution thermal flux 108 with those of the meridians 116 to provide an overall thermal view of the human subject for the purposes of providing health evaluation and/or medical diagnosis on the human subject.

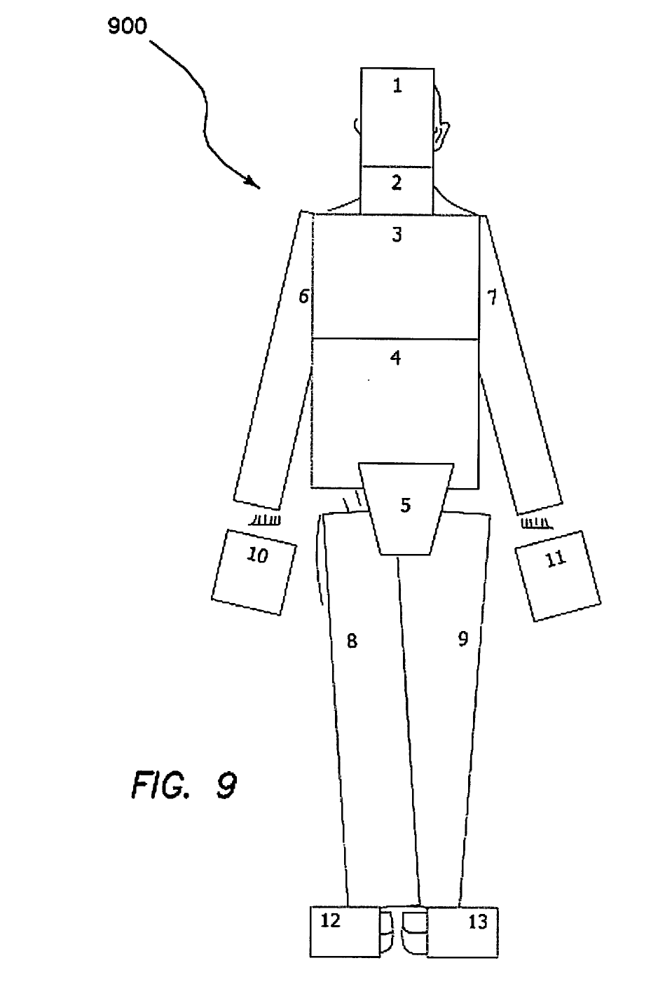

Reference is now made to FIG. 9 illustrating a perspective view of the various regions of the human subject 900 for purposes of dividing the body into different regions for calculation and analysis of the meridians, channels and vessels 116. In this example, the body can be divided into 13 different regions. The first section includes the head, the second section being the neck, third section being the thorax, fourth section being the abdomen, fifth section being the pelvis, sixth and seventh sections being the right and left arms, respectively, eighth and ninth sections being the right and left hands, respectively, tenth and eleventh sections being the right and left legs, respectively, and the twelfth and thirteenth sections being the right and left foot, respectively. Although the regions of the human body 900 have been divided into thirteen regions, it will be appreciated by one skilled in the art that the body 900 can be divided into more or fewer regions. The geometric ratios of each region's bone structures and internal organs can be combined with information from adjacent regions to derive the one or more meridians traveling throughout the human subject as discussed before in accordance with traditional Chinese medicine principles. And like above, the various regions can utilize images from thermal scanning or other scanning processes. The geometric derivation for the meridians 116 may also include identifiable thermal features including the likes of the navel.

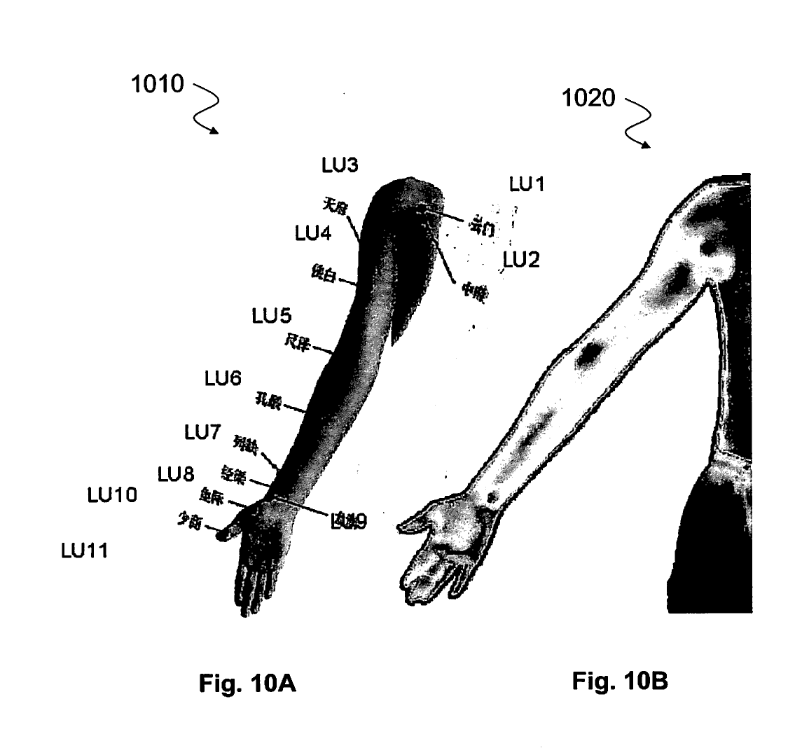

Reference is now made to FIGS. 10A-10B illustrating perspective views of the process of acquiring the positions or acupoints of the lung channel of hand taiyin by correlating the thermal image 1020 with that of traditional Chinese medicine principle 1010. As shown in the traditional layout of the acupoints for the lung channel of hand taiyin 1010, there are approximately 11 pressure points in accordance with traditional Chinese medicine theory as illustrated by labels LU1 through LU11. The pressure points extend the entire length of the arm from the shoulder area to the thumb. By knowing the position of these channels in accordance with the geometric dimension of the arm, shoulder, fingers and other parts of the body, a calculated spatial distribution of the heat flux from the body can be correlated with those of the channels to better provide for a proper health evaluation and medical diagnosis of the patient. The paths of the meridian (not shown in this instance) may also be correlated to the spatial distribution heat flux map 1020 and studied and analyzed in accordance with traditional Chinese medicine theory.

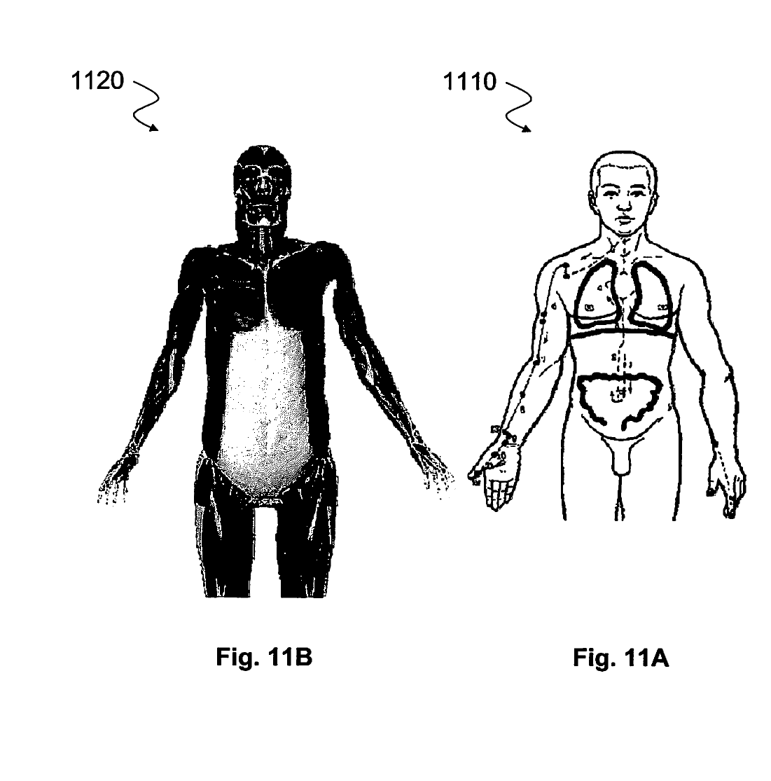

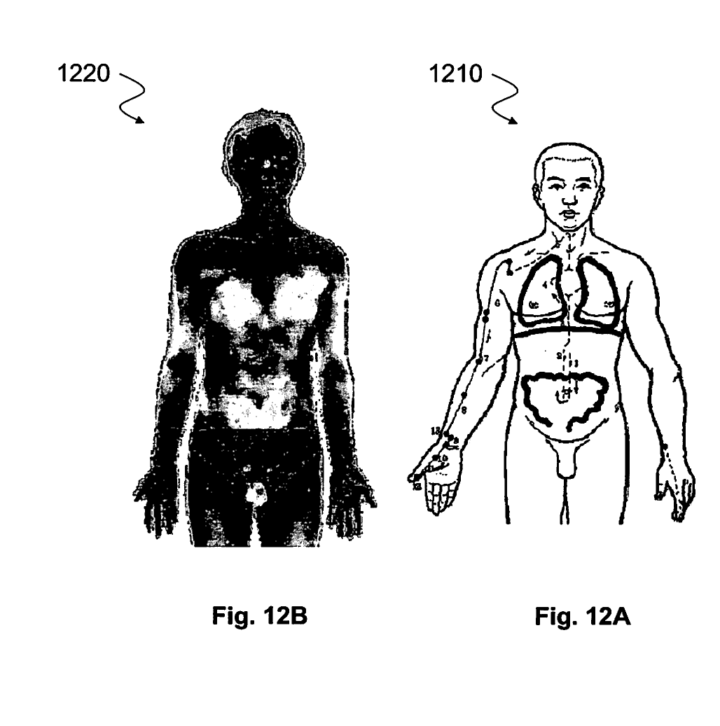

Reference is now made to FIGS. 11B-12B further illustrating the processes involved in calculating the meridians for the comparison and analysis step. FIG. 11A illustrates a physical model of the lung channel of hand taiyin 1110 in accordance with traditional Chinese medicine theory while FIG. 11B illustrates a skeletal muscle system showing the locations of the anatomical muscles and body parts. By comparing the two figures, automatic electronic correlations can be taken into account to determine proximate areas of where the acupoints for the lung channel of hand taiyin 1110 correlate with which muscles of the human body. Additionally, FIG. 12A illustrates the same physical model of the lung channel of hand taiyin 1210 while FIG. 12B is that of a spatial distribution thermal map 1220 of a human subject. In conjunction with the previous comparison and analysis, determinations can be made in several respects. First, the acupoints of the lung channel of hand taiyin may be determined and correlated with the spatial distribution points. Second, the determined points may be correlated with the skeletal muscles to better evaluate where the ailments may be located. Although the skeletal muscle system is shown and described, it is understood that other systems of the human body like the circulatory and digestive systems may be incorporated.

Case Studies

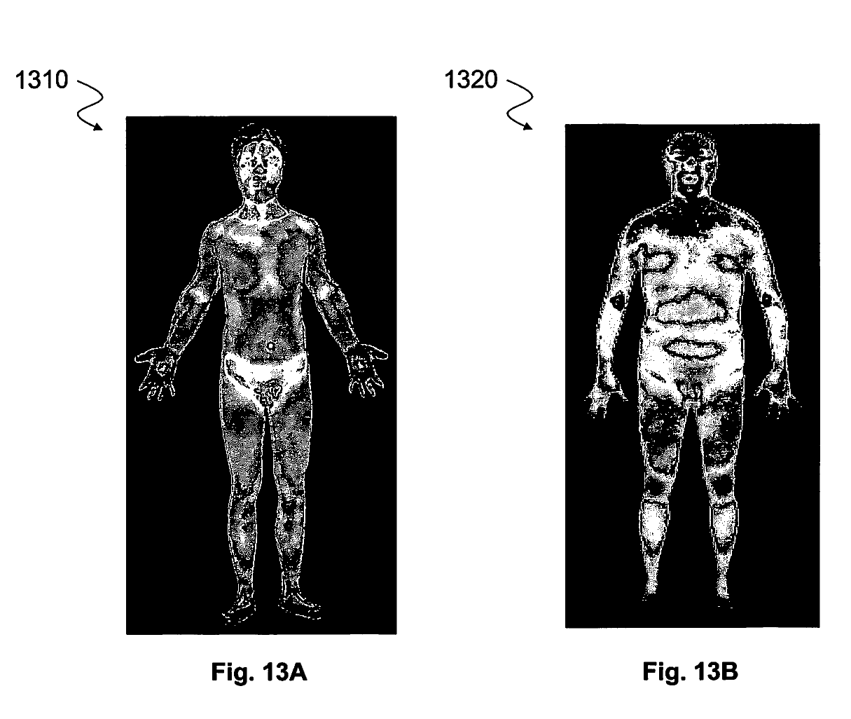

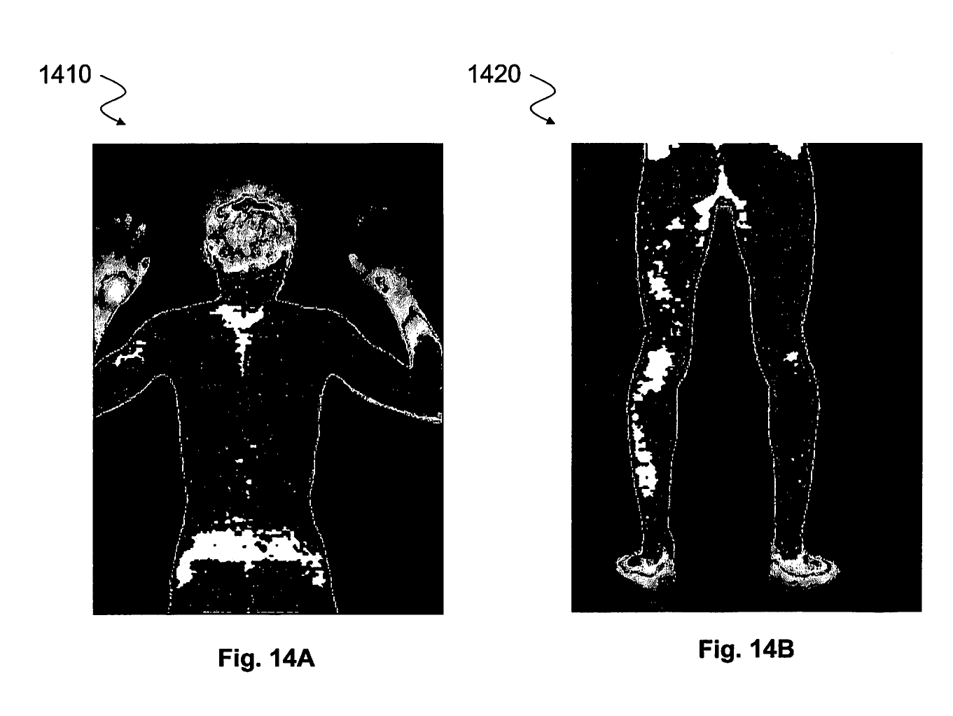

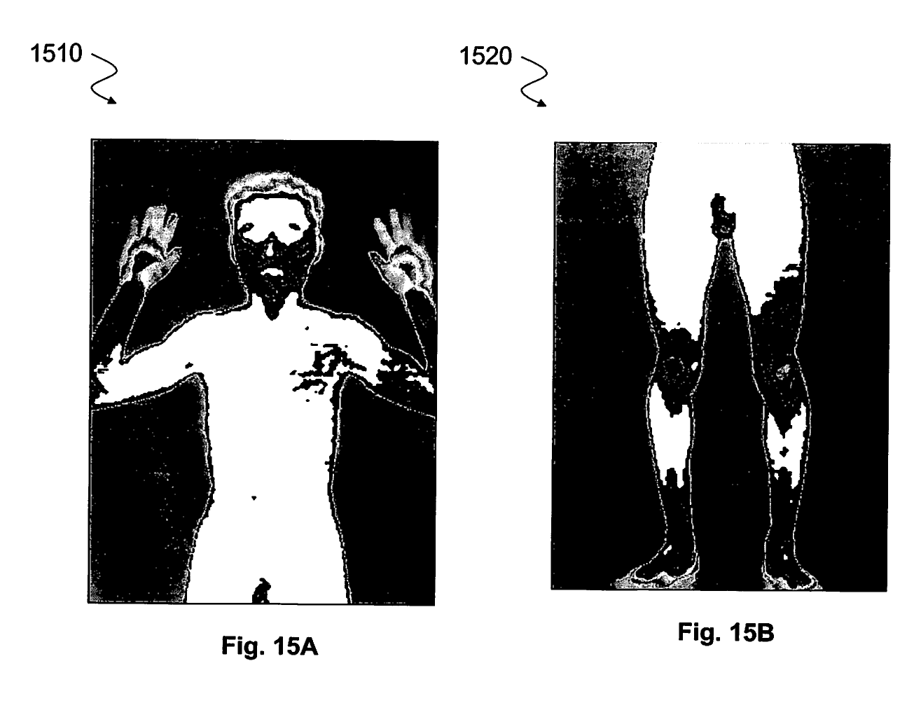

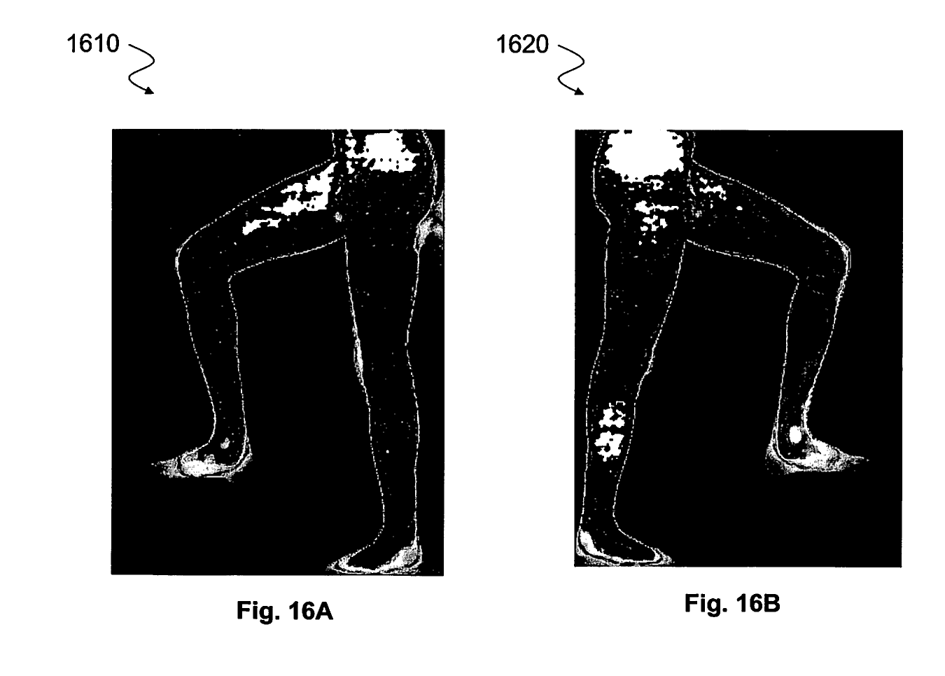

Reference is now made to FIGS. 13B-16B illustrating imaging results of two substantially healthy human subjects 1310, 1320. As shown in FIGS. 13A and 13B, the spatial distribution thermal maps 1310, 1320 of the two individuals exhibit substantially uniform hot and cold regions evenly distributed and bilaterally symmetrical from head to the toe. FIGS. 14B and 15B illustrate posterior and frontal views of the human subject, respectively, with similar findings where there are no substantial hot spots or flares on the spatial distribution thermal maps 1410, 1420, 1510, 1520. Likewise, FIG. 16A illustrates the human subject in a standing position with the right leg slightly elevated 1610, while FIG. 16B illustrates the similar standing position with the left leg slightly elevated 1620. Doing a side-by-side comparison and additional in-depth analysis, it is evident that the hot and cold regions of the human subject's legs are evenly distributed and substantially bilaterally symmetrical indicative of no major blockage or heat build up anywhere about the body.

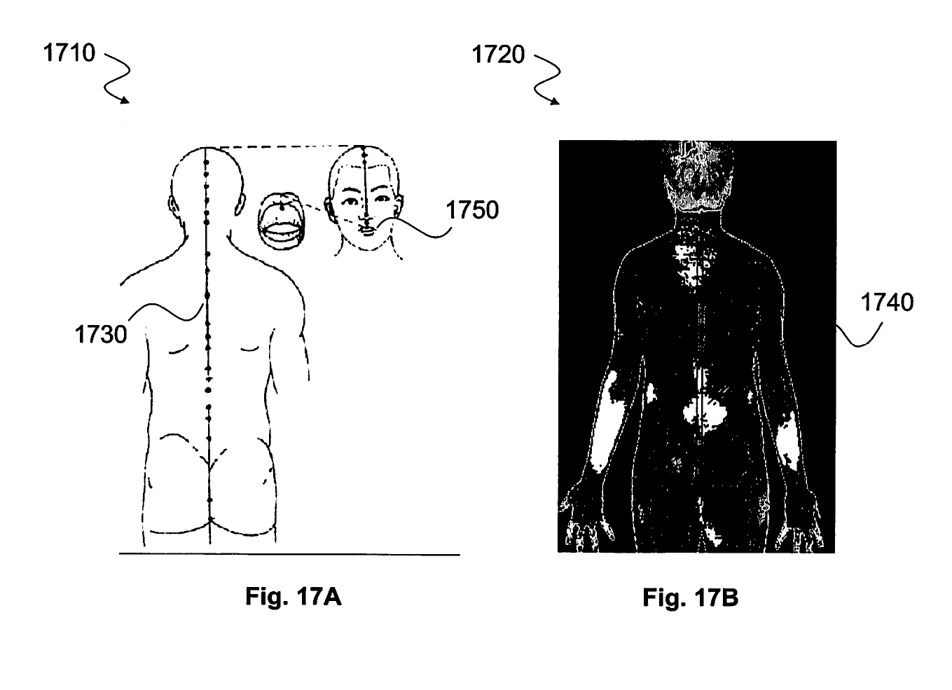

Reference is now made to FIGS. 17B-23B illustrating imaging results of a case study involving a 14-year-old patient utilizing the disclosed health evaluation and medical diagnosis system and method. The patient presents with fever of unknown origin. Antibiotics did not result in any substantial signs of improvement. Nothing unusual was observed on chest x-rays or CT scans. Initial diagnosis suggested pulmonary interstitial pneumonia. The patient's axillary temperature measured 42° C. while mouth and rectal temperatures were normal. Nothing strange was observed in terms of eating, drinking, sleeping, weight gain and defecation during the duration of the fever. Tuberculosis, typhoid fever, bacillary dysentery or other infectious diseases were not suspected. Chronic diseases such as rheumatoid or other diseases that the patient may have come in contact with were ruled out. During preliminary examination, nothing unusual was observed other than some tenderness in the upper abdominal area. Routine blood work showed blood, urine, hematuria and blood biochemistry panels were all within normal range. Immunoglobulin levels were also normal as was the EEG scan. Bilateral neck and associated lymph nodes were normal with slightly increased liver and spleen. PET scan revealed that groin and neck areas including pulmonary lymph nodes exhibited increased metabolism leading to slightly enlarged liver and spleen.

FIG. 17A illustrates a perspective view of an energy map 1710 of the governor vessel 1730 in accordance with traditional Chinese medicine theory, while FIG. 17B illustrates a spatial distribution thermal map 1720 as acquired by processing a thermal image and deriving the temperature properties. In looking at the governor vessel 1730 (also referred to as the ren meridian), we can see that the main portion of this meridian or channel 1730 ascends along the midline of the back to the top of the head, and then descends along the midline of the face down GV28 1750, a point between the upper lip and the upper gum in the labia frenum, also called governing vessel 1750. By doing a side-by-side comparison of the two images 1710, 1720, the spatial distribution 1720 as measured from the patient shows some hot spots 1740 indicative of blockage and heat build up approximately near the lower back area. Further comparison and analysis of the spatial distribution thermal map 1720 with derived meridians and channels 1710 has identified blockage and heat build up along various channels or points (GV2, GV4-GV5 and GV12) of the governor vessel 1730.

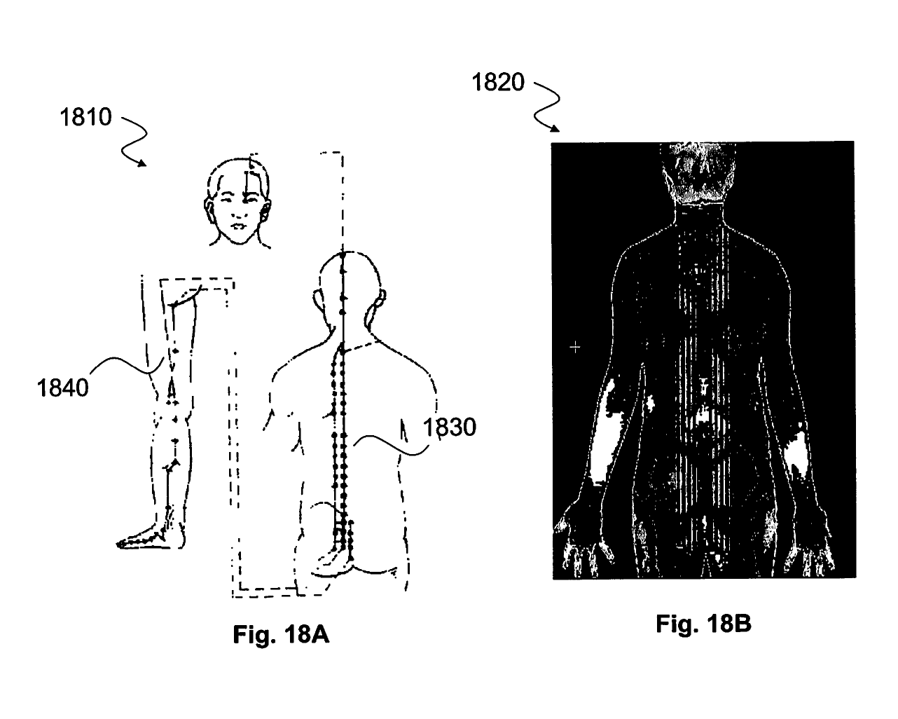

FIG. 18A illustrates a perspective view of an energy map of the bladder meridian 1810 in accordance with traditional Chinese medicine theory, while FIG. 18B illustrates a spatial distribution thermal map 1820 as acquired by processing a thermal image and deriving the temperature properties. The bladder meridian foot taiyang 1810 includes multiple branches 1830 descending a person's backside continuing along the latero-posterior side of the thigh 1840. By doing a side-by-side comparison and additional in-depth analysis of the spatial distribution 1820 with derived meridians and channels 1810, the blockage and heat build up is detected along various channels or points (BL11, BL12/41, BL22/51, BL23/52 and BL25) of the bladder meridian 1830, 1840.

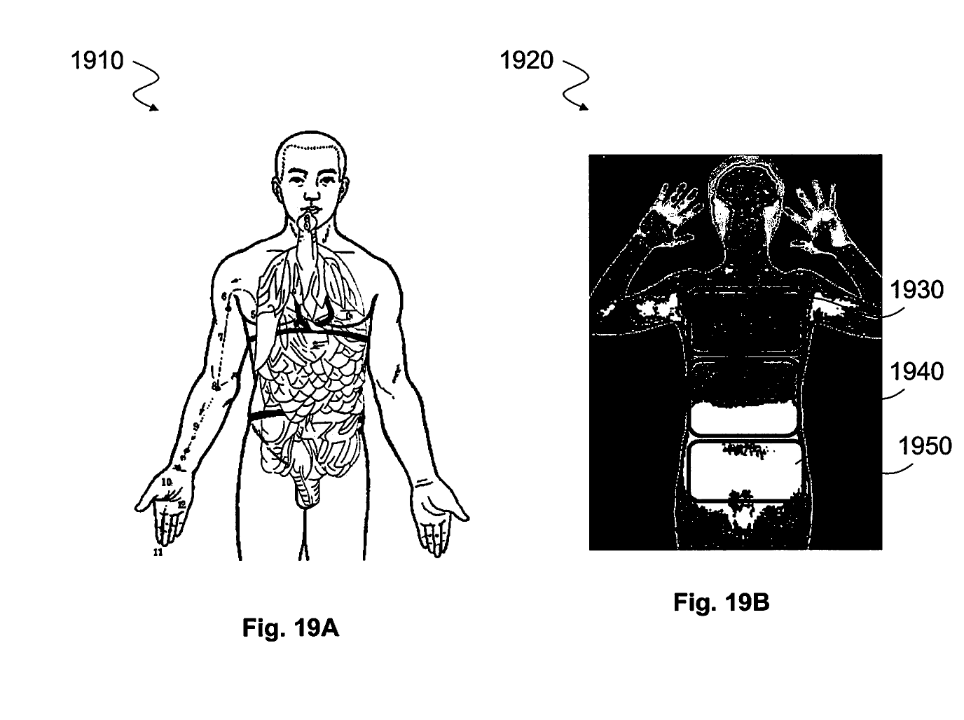

FIG. 19A illustrates a perspective view of an energy map of the san jiao meridian 1910 in accordance with traditional Chinese medicine theory, while FIG. 19B illustrates a spatial distribution thermal map 1920 as acquired by processing thermal images and deriving temperature properties. As discussed above, the san jiao meridian 1910 includes three compartments 1930, 1940, 1950 and can sometimes be referred to as the triple heater or triple energizer organs. By doing a side-by-side comparison and further in-depth analysis of the spatial distribution 1920 with derived channels 1910, it is observed that blockage and heat build up can be seen in the middle 1940 and lower 1950 compartments of the san jiao meridian, but the upper compartment 1930 did not exhibit any abnormal heat distribution as determined in accordance with traditional Chinese medicine theory.

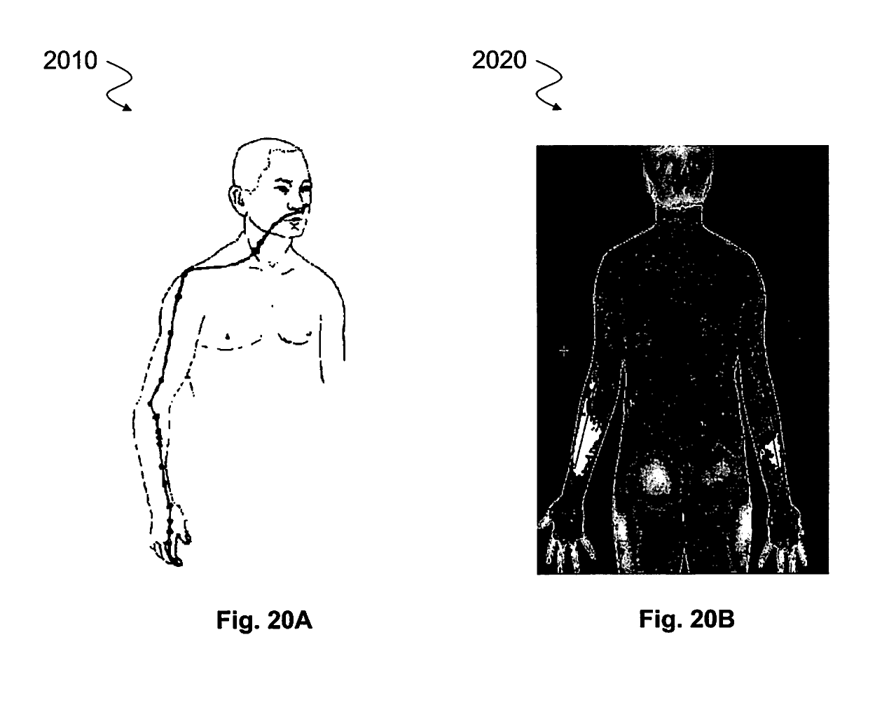

FIG. 20A illustrates a perspective view of an energy map of the large intestine meridian 2010 in accordance with traditional Chinese medicine theory, while FIG. 20B illustrates a spatial distribution thermal map 2020 as acquired by processing a thermal image and deriving the associated temperature properties. The large intestine meridian of hand yangming 2010 includes energy or acupoints extending throughout the lateral side of the arm all the way up to the head area. The meridian 2010 also includes acupoints on the other side of the body (not shown). By doing a side-by-side comparison and further in-depth analysis of the spatial distribution 2020 with the derived channels 2010, it was proposed that blockage or abnormal heat distribution was detected along various channels or points (LI11 and LI10-9-8-7-6) of the large intestine meridian.

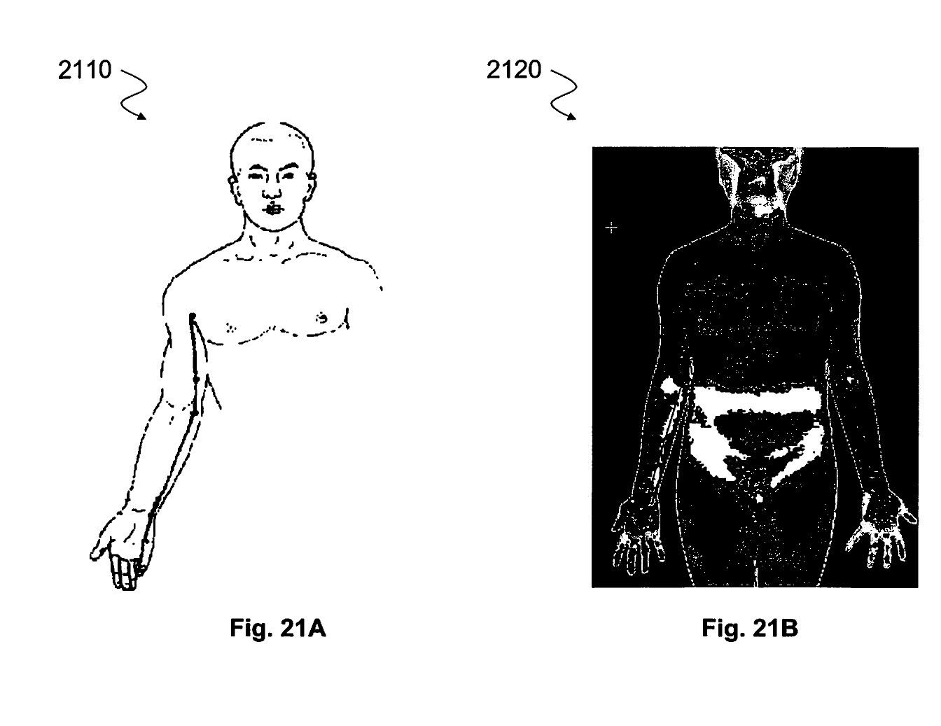

FIG. 21A illustrates a perspective view of an energy map of the heart meridian 2110 in accordance with traditional Chinese medicine theory, while FIG. 21B illustrates a spatial distribution thermal map 2120 as acquired by processing a thermal image and deriving the associated temperature properties. The heart meridian of hand-shaoyin 2110 includes energy or acupoints extending through the ulnar side of the forearm medially. By doing a side-by-side comparison and further in-depth analysis of the spatial distribution 2120 with the derived channels 2110, blockage or abnormal heat distribution can be observed along various points (HT3-4-5-6) of the heart meridian. Further, it can also be observed that an imbalance exist between the left and right forearms of the human subject by cursory examination of the spatial distribution thermal map 2120.

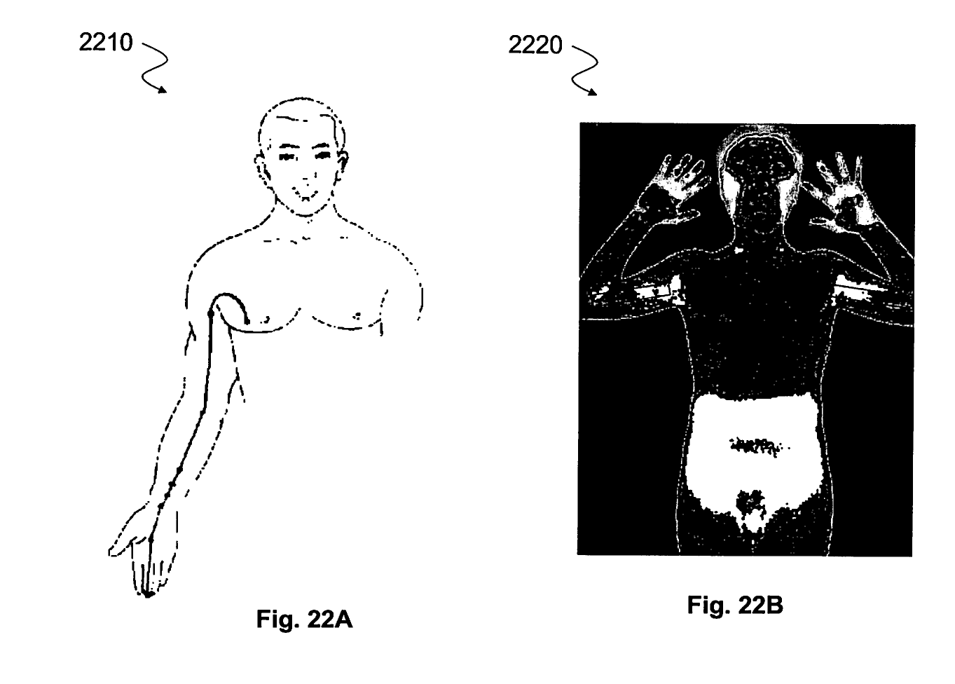

FIG. 22A illustrates a perspective view of an energy map of the pericardium meridian 2210 in accordance with traditional Chinese medicine theory, while FIG. 22B illustrates a spatial distribution thermal map 2220 as acquired by processing a thermal image and deriving the associated temperature properties. The pericardium meridian of hand-jue yin 2210 includes energy or acupoints similar to that of the heart meridian but further extends to the chest region of the patient. By doing a side-by-side comparison and further in-depth analysis of the spatial distribution 2220 with the derived channels 2210, it was proposed that blockage or abnormal heat distribution was detected along various channels or points (PC2 and PC3) of the pericardium meridian.

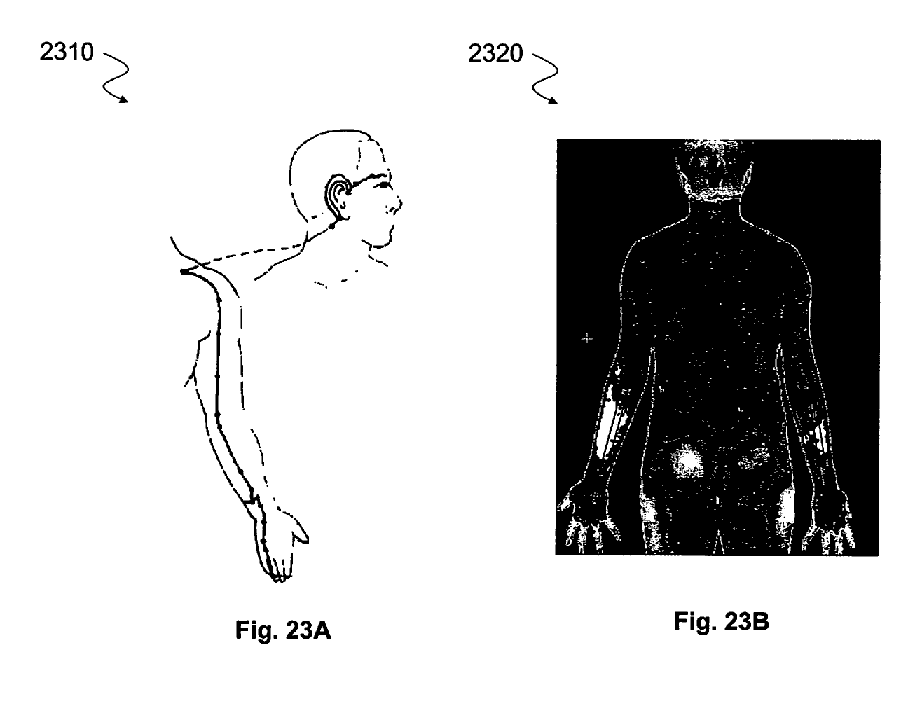

FIG. 23A illustrates a perspective view of an energy map of the san jiao meridian of hand-shaoyang 2310 in accordance with traditional Chinese medicine theory, while FIG. 23B illustrates a spatial distribution thermal map 2320 as acquired by processing a thermal image and deriving the associated temperature properties. The san jiao meridian of hand-shaoyang 2310 includes energy or acupoints that are substantially similar to those of the large intestine meridian. By doing a side-by-side comparison and further in-depth analysis of the spatial distribution 2320 with the derived channels 2310, blockage or abnormal heat distribution can be observed along various points (SJ9-SJ8-SJ7/6-SJ5) of the san jiao meridian of hand-shaoyang.

Once blockages and locations where the heat dissipations are not taking place are identified, treatments for these specific blockages or lack of flow can be accomplished with the appropriate Chinese medicine, Western medicine or a combination thereof. In summary, the embodiments of the present invention provide an automated, Chinese medicine standardized assessment capable of determining spatial location and description of energy points within a human subject in accordance with traditional Chinese medicine theory. Based on traditional Chinese medicine theory, healthy organs including internal organs and visceral organs connected with meridians, channel points and vessels do not exhibit abnormal energy because qi or life force energy flows freely within. However, if there is any abnormality or disturbance of the human body due to ailments or other external forces, the assessment is able to provide a quantitative analysis of the abnormal energy as it builds up or accumulates at or near the point of abnormality or disturbance. In other instances, the abnormal energy may present on the surface of the body and can also be readily determined by the assessment method.

Some of the features of the embodiments of the present invention include:

(1) Sensitivity (early, pre-discovery): Thermal imaging scans of the human body at early stages can detect changes in cell metabolism and lead to as early detection of diseases as possible.

(2) Specificity (identification assessment): Thermal imaging scans provide multiple sets of data and information for making comprehensive assessment of the human subject's health status and therefore, high specificity.

(3) Integrity: The comprehensive inspection of a human subject's body metabolism and health status can be provided in a short period of time, which is consistent with the overall view of Chinese medicine.

(4) Fast and efficient: Complete thermal scans of a human subject can be rapidly carried out to provide a plurality of health-related data thereby allowing a typical clinic to see multiple patients each day. In some instances, medical diagnosis including treatment methods and techniques and possible side effects can be provided within 60 minutes from the time the infrared images are acquired.

(5) Network and information: Typical case needs about 10 frames of infrared images occupying kilobits or megabits. With high speed Internet access, information can be rapidly shared among medical personnel and patients.

(6) With high temperature resolution, spatial resolution and rich image processing functions, analysis and diagnosis of patient data can be intuitive and convenient.

Although the invention has been described in detail with reference to several embodiments, additional variations and modifications exist within the scope and spirit of the invention as described and defined in the following claims.

What is claimed is:

1. A method comprising:
acquiring one or more images of a human subject;
defining one or more bone structures of the human subject using the one or more images;
identifying one or more parts of the human subject based on the one or more bone structures and the one or more images; and
establishing one or more meridians and vessels of the human subject based on geometric ratios between the bone structures and parts of the human subject.

2. The method of claim 1, further comprising analyzing one or more temperature features from the one or more images for identifying the one or more parts of the human subject.

3. The method of claim 2, further comprising dissecting the one or more images of the human subject into one or more regions to facilitate defining the one or more bone structures of the human subject.

4. The method of claim 3, further comprising manually or automatically executing the steps.

5. A computer system comprising:
means for defining one or more bone structures of a human subject from one or more images of the human subject;
means for identifying one or more parts of the human subject based on the one or more bone structures; and
means for establishing one or more viscera and bowels, meridians and vessels and acupoints of the human subject based on geometric ratios between the bone structures and parts of the human subject.

6. The system of claim 5, further comprising means for analyzing one or more temperature features from the one or more images for identifying the one or more parts of the human subject.

7. The system of claim 5, further comprising means for dissecting the one or more images of the human subject into one or more regions to facilitate defining the one or more bone structures of the human subject.

8. The system of claim 5, further comprising means for storing the one or more viscera and bowels, meridians and vessels and acupoints on an electronic medium.

9. The system of claim 5, wherein the computer system is automated.

10. A method comprising:
acquiring one or more thermal points of a human subject;
calculating spatial distribution of the one or more thermal points to provide one or more energy points of the human subject; and
comparing the one or more energy points with one or more viscera and bowels, meridians and vessels and acupoints of the human subject to provide a health evaluation of the human subject.

11. The method of claim 10, further comprising acquiring one or more thermal images of the human subject using one or more imaging devices.

12. The method of claim 11, further comprising converting the one or more thermal images into the one or more thermal points of the human subject.

13. The method of claim 10, further comprising performing data processing on the one or more thermal points of the human subject.

14. The method of claim 13, further comprising utilizing data processing having signal smoothing and improving signal to noise ratio of the one or more thermal points.

15. The method of claim 10, further comprising displaying the one or more thermal points as one or more temperature field maps.

16. The method of claim 10, further comprising analyzing the one or more energy points with the one or more viscera and bowels, meridians and vessels and acupoints to provide a medical diagnosis of the human subject.

17. The method of claim 10, further comprising pre-determining the one or more viscera and bowels, meridians and vessels and acupoints from one or more images of the human subject.

18. The method of claim 10, further comprising pre-storing the one or more viscera and bowels, meridians and vessels and acupoints in one or more databases.

19. The method of claim 10, wherein the one or more energy points include one or more qi points.

20. The method of claim 10, further comprising manually or automatically executing the steps.

21. A computer system comprising:
means for receiving one or more thermal points of a human subject;
means for calculating spatial distribution of the one or more thermal points to provide one or more energy points of the human subject; and
means for comparing the one or more energy points with one or more viscera and bowels, meridians and vessels and acupoints of the human subject to provide a health evaluation of the human subject.

22. The system of claim 21, further comprising means for receiving one or more thermal images of the human subject acquired using one or more imaging devices.

23. The system of claim 22, further comprising means for converting the one or more thermal images of the human subject into the one or more thermal points.

24. The system of claim 21, further comprising means for performing data processing of the one or more thermal points of the human subject.

25. The system of claim 24, wherein the means for performing data processing includes signal smoothing and signal to noise ratio improvement of the one or more thermal points.

26. The system of claim 21, further comprising means for displaying the one or more thermal points of the human subject as one or more temperature field maps.

27. The system of claim 21, further comprising means for analyzing the one or more energy points with the one or more viscera and bowels, meridians and vessels and acupoints of the human subject to provide a medical diagnosis of the human subject.

28. The system of claim 21, wherein the one or more meridians and vessels are pre-determined from one or more images of the human subject.

29. The system of claim 21, wherein the one or more viscera and bowels, meridians and vessels and acupoints are pre-stored on one or more databases.

30. The system of claim 21, wherein the one or more energy points include one or more qi points.

31. The system of claim 21, further comprising means for storing the one or more viscera and bowels, meridians and vessels and acupoints on an electronic medium.

32. The system of claim 21, wherein the computer system is automated.

33. A computer system comprising:
a first module adaptable to define one or more bone structures of a human subject from one or more images of the human subject;
a second module capable of identifying one or more parts of the human subject based on the one or more bone structures; and
a third module configured to establish one or more viscera and bowels, meridians and vessels and acupoints of the human subject based on geometric ratios between the bone structures and parts of the human subject.

34. The system of claim 33, wherein the first, second and third modules are integrated in an application.

35. The system of claim 33, further comprising a fourth module adaptable to analyze one or more temperature features from the one or more images for identifying the one or more parts of the human subject.

36. The system of claim 33, further comprising a fifth module capable of dissecting the one or more images of the human subject into one or more regions to facilitate defining the one or more bone structures of the human subject.

37. The system of claim 33, further comprising an electronic storage medium for storing the one or more viscera and bowels, meridians and vessels and acupoints.

38. The system of claim 33, wherein the computer system is automated.

39. A computer system comprising:
a first module adaptable to receive one or more thermal points of a human subject;
a second module configure to calculate spatial distribution of the one or more thermal points to provide one or more energy points of the human subject; and
a third module capable of comparing the one or more energy points with one or more viscera and bowels, meridians and vessels and acupoints of the human subject to provide a health evaluation of the human subject.

40. The system of claim 39, wherein the first, second and third modules are integrated in an application.

41. The system of claim 39, further comprising a fourth module configured to receive one or more thermal images of the human subject acquired using one or more imaging devices.

42. The system of claim 39, further comprising a fifth module adaptable to convert the one or more thermal images of the human subject into the one or more thermal points.

43. The system of claim 39, further comprising a sixth module capable of performing data processing of the one or more thermal points of the human subject including signal smoothing and signal to noise ratio improvement of the one or more thermal points.

44. The system of claim 39, further comprising a seventh module capable of displaying the one or more thermal points of the human subject as one or more temperature field maps.

45. The system of claim 39, further comprising an eighth module adaptable to analyze the one or more energy points with the one or more viscera and bowels, meridians and vessels and acupoints of the human subject to provide a medical diagnosis of the human subject.

46. The system of claim 39, wherein the one or more meridians and vessels are pre-determined from one or more images of the human subject.

47. The system of claim 39, wherein the one or more viscera and bowels, meridians and vessels and acupoints are pre-stored on one or more databases.

48. The system of claim 39, wherein the one or more energy points include one or more qi points.

49. The system of claim 39, further comprising an electronic storage medium for storing the one or more viscera and bowels, meridians and vessels and acupoints.

50. The system of claim 39, wherein the computer system is automated.
